(19) United States
(12) Patent Application Publication
(10) Pub. No.: US 2017/0354168 A1
(43) Pub. Date: Dec. 14, 2017

(54) Feeding Algae to Cattle at Low Doses to Produce High Omega 3 Levels in Beef

(71) Applicant: Donald M Smith, Aspen, CO (US)

(72) Inventor: Donald M Smith, Aspen, CO (US)

(21) Appl. No.: 15/520,997

(22) PCT Filed: Oct. 21, 2015
(86) PCT No.: PCT/US2015/056695

§ 371 Date: Apr. 21, 2017

Related U.S. Application Data
(60) Provisional application No. 62/066,778, filed on Oct. 21, 2014.

(51) Int. Cl. A23K 50/10; A61K 31/202; A23K 10/30; A61K 36/02; A23K 10/16

(52) U.S. Cl. CPC A23K 50/10; A61K 36/02; A23K 10/16; A23K 10/30; A61K 31/202

(57) ABSTRACT

Disclosed herein is a method for feeding low doses of algae to cattle to increase the level of omega 3 polyunsaturated fatty acids including docosahexaenoic acid (DHA) and eicosapentenoic acid (EPA) in commercial cattle feeding environments. The method of increasing the level of omega-3 fatty acids in cattle which comprises feeding the cattle between about 0.05 and 0.8 pounds per day of algae.

### BACKGROUND OF THE INVENTION

Cardiologists encourage consumers to eat fish daily to have a high intake of the omega 3s DHA (docosahexaenoic acid) and EPA (eicosapentaenoic acid). Omega 3 fatty acids, particularly DHA and EPA, support cardiovascular, brain, and eye health and provide additional health benefits. However the daily consumption of fish also provides the consumer with the bad health consequences of mercury, other heavy metals, and PCB's. In addition, the world's supply of fish is declining due to overfishing. Fish farming intends to substitute for the decline in ocean fish populations, but puts increasing pressure on the supply of baitfish. Fish farmers must use fishmeal obtained by netting huge quantities of small fish in the ocean, i.e. baitfish. The baitfish contain omega 3 fatty acids in their fat tissues which are deposited when these smaller fish eat zooplankton. The zooplankton eat algae and absorb the omega 3 fatty acids from the algae as it is the base of the omega 3 food chain. The fish meal is needed in fish farming to supply the omega 3's to the larger farmed fish.

In sum, netting baitfish for fishmeal for aquaculture is not sustainable in the long term. New methods to get more omega 3s in the human diet are needed. New, cost-effective ways of producing omega 3s in natural foods are particularly needed.

Algae, which normally occur in the ocean and in fresh water, are the ultimate source of omega 3s DHA and EPA. Most algae use sunlight in photosynthesis to convert CO2 and H2O to carbohydrates, which are processed within the algae into lipids, which are further processed into the polyunsaturated omega 3 fatty acids, in particular DHA and EPA. Some algae may also consume sugars directly, and then process the sugars into lipids and omega 3s. Many companies have developed ways to grow algae on land in open ponds, or in containment systems to grow algae in sanitary conditions. Some companies have developed ways of feeding sugar to algae in closed tanks, usually in a fermentation process.

Most algae feeding inventions are designed to feed very small amounts to increase the health of the cattle. Alternatively, omega 3s are introduced from fish meal, after the fish meal is encapsulated, so that the omega 3s are protected from biohydrogenation (processed into saturated fats) in the rumen (stomach). U.S. Pat. No. 8,797,916 of Smith discloses the use of whole microalgae to feed to cattle in relatively large amounts. The Smith '916 patent discloses the optimum amount for feeding cattle to obtain the highest amounts of DHA and EPA in the beef is between about 0.8 to 1.2 pounds of algae per day in a feedlot ration containing significant amounts of corn. The amount of algae (which is high in fat) that a cow will ingest per day is limited to an amount between the amount of fat in the ration with corn, and the maximum fat the cattle will eat. Since cattle self-regulate the amount of fat they will consume as a percentage of their total feed ration to 8%, and since corn contains almost 6% fat, Smith discloses that substituting other lower fat grains for corn will allow feeding more algae. Wheat or barley contain about 36% as much fat as corn. Thus Smith discloses that substituting wheat or barley for whole corn will allow the feeding of about 2 to 3 pounds of algae per day, while maintaining the same total feed intake by the cattle.

The Smith '916 patent maximizes the level of omega 3 fatty acid content in beef. It has now been discovered that a lower dose of algae in the feed ration of cattle can produce very significant amounts of omega 3 fatty acids in the beef, and cost effectively. The levels are below the maximum produced by the method disclosed in the Smith '916 patent, but do so more efficiently. That is, the new methods produce omega 3s in the beef with less input of algae, i.e. at a lower cost per milligram of omega 3 fatty acids.

### DETAILED DESCRIPTION OF THE INVENTION

Previously disclosed levels of feeding algae to cattle produced very high amounts of omega 3s DHA and EPA in the beef. However the cost of this feeding was relatively high. Feeding 1 pound of algae for 30 to 90 days, for example costs approximately $150 to $450, which adds approximately $0.37 to $1.12 per pound to the production cost of boned meat (hereinafter referred to as Omega3Beef).

It has now been discovered that feeding a smaller ration of algae to cattle as a supplement to their feed can produce beef containing high levels of omega 3 fatty acids at a significantly lower cost per unit of omega 3 per pound of beef. Feeding cattle between about 0.05 and 0.8 pounds of algae per day and more preferably between about 0.2 and 0.6 pounds of algae per day, and most preferably between about 0.2 and 0.4 pounds of algae per day results in omega 3 beef having higher levels of omega3 fatty acids and significantly reduces the cost of omega3 fatty acids content per pound of the beef.

The following feeding trials illustrate the invention. These are examples and do not limit the invention.

TABLE 1
Alternate Algae Doses and Alternate Omega 3 Levels in Beef
(DHA and EPA in mg. per 8 oz. steak)

| | | Total | Total | | | | | | Cost |
|---|---|---|---|---|---|---|---|---|---|
| | | Algae | Cost | | | | DHA + EPA | Cost of | of |
| | Supplement | per | of | | | | (mg.)/lb. | algae/mg. | algae |
| | feeding | plan- | Algae | DHA | EPA | DHA + EPA | of algae | DHA + EPA | per lb. |
| Line | plan | (lbs.) | ($) | (mg.) | (mg.) | (mg.) | fed | deposited. | beef |
| 1. | 0.6-1.2 lbs@ | 72 | $360 | 47 | 94 | 141 | 1.9 | $2.55 | $0.90 |
| | 90 days | | | | | | | | |
| 2. | 0.4 last 30 | 12 | $60 | 31 | 27 | 58 | 4.8 | $1.03 | $0.15 |
| | days | | | | | | | | |
| 3. | 0.2-.04 lbs | 24 | $120 | 31 | 50 | 81 | 3.4 | $1.48 | $0.30 |
| | @ 90 days | | | | | | | | |
| 4. | 0.4-0.8 lbs | 48 | $240 | 41 | 81 | 125 | 2.6 | $1.92 | $0.60 |
| | @ 90 days | | | | | | | | |

Line 1, Table 1, shows that maximizing omega 3s DHA and EPA in the beef is accomplished by feeding the cattle for 60 days (days 90 through 31 before slaughter) at 0.6 pounds of algae per day, and then 1.2 pounds of algae per day for the last 30 days just before slaughter. This results in 141 mg of DHA plus EPA per 8 ounce portion of steak. However only 1.9 mg of DHA&EPA are produced per pound of algae fed over the 90 days. The cost of algae per mg of DHA plus EPA in the steak is $2.55.

PROTOCOL—A preferred embodiment of the invention is illustrated in the experiment reported on Line 2 of table 1: Line 2, Table 1, shows that feeding 0.4 pounds of algae only for the last 30 days just before slaughter results in 58 mg of DHA&EPA per 8 oz. portion of steak. 4.83 mg of DHA&EPA are produced per pound of algae fed over the 30 days. The cost of algae is $1.03 per mg of DHA&EPA in the steak.

Line 4, Table 1 shows that feeding 0.4 pounds of algae per day for 60 days, followed by feeding 0.8 pounds per day for the last 30 days results in 125 mg of DHA&EPA in an 8 oz. portion of steak. The cost of algae is $1.92 per mg of DHA&EPA.

Another preferred embodiment of the invention is disclosed in the feeding trial reported at Line 3 of table 1: Line 3 of table 1 shows that feeding 0.2 pounds of algae for 60 days followed by feeding 0.4 pounds of algae per day for the last 30 days results in 81 mg. of DHA plus EPA. The cost of algae is $1.48 per mg of DHA&EPA in an 8 oz. serving of steak.

The data points produced in the above examples may be used to predict scientifically the effects of even lower doses. A dose of 0.1 pound per day over 30 to 90 days is expected to produce EPA plus DHA of 25 mg in an 8 oz. portion of steak. A dose of 0.05 pounds per day over 30 to 90 days is predicted to produce EPA plus DHA of 14 mg.

Whereas my prior patent (U.S. Pat. No. 8,747,916) discloses feeding algae at 0.8 to 1.2 pounds per day, the present invention indicates that dosages as low as 0.05 pounds per day are extremely effective to create high levels of omega 3s in beef at lower cost and by feeding less algae for each milligram of omega 3s created in the beef. Based on the data points above, feeding amounts as low as 0.1 pounds per day will produce a measurable increase in DHA plus EPA per steak, and amounts as low as 0.05 pounds per day will still produce measurable amounts.

The present invention establishes that there are more cost effective algae feeding protocols then the one which maximizes the amount of EPA&DHA in the beef. Feeding algae at between 0.8 and 1.2 pounds per day for 90 days (total 72 pounds of algae) requires 0.51 pounds of algae to produce 1 mg of DHA plus EPA in an 8 ounce portion of steak (Line 1 above). In contrast, feeding between about 0.2 and 0.4 pounds per day of algae for 90 days (line 3 above) (total 24 pounds of algae) requires 0.296 pounds of algae to produce 1 mg of DHA plus EPA in an 8 ounce portion of steak. And feeding 0.4 pounds for the last 30 days only requires 0.20 pounds to produce 1 mg. of DHA plus EPA in an 8 oz. portion of steak (Line 2 above).

Various new products advertise their high omega 3 content. Research indicates that regular feedlot produced beef contains about 7 mg of DHA plus EPA per 8 oz. portion of steak. Grass fed beef produce about 14 mg of DHA plus EPA. At the other extreme halibut produce about 295 mg, and natural salmon contain about 500 to 800 mg of DHA plus EPA per 3.5 oz. portion.

Omega 3 eggs, which are fed the same algae used in the Smith '916 patent and the current invention, contain about 180 mg of DHA plus EPA. A commercially available product (Horizon Organic milk) rich in omega 3s contains about 32 mg of DHA per 8 oz. serving. The DHA oil, which is mixed into the milk has been extracted from the same species of algae used in the Smith 916 patent and the current invention. At the lowest extreme, one high-end supermarket chain sells a chocolate bar advertising the omega 3 DHA content as about 12 mg. per portion, where extracted DHA oil has been mixed in the candy from the same type of algae used in the method of the present invention.

The preferred algae for use in the present invention is Schizochytrium but any other algae: 1. having a cell wall that is relatively indigestible in the rumen; 2. with a high amount of DHA and/or EPA by weight (e.g. greater than 10%); and 3. a high proportion of EPA plus DHA by weight of total fats (e.g. 20-50%) can be used in practicing the present invention. The percentage of algal cell walls that survive passage through the rumen of the intestine will vary depending on the contents of the rumen and the diet of each animal, however in general the majority of the omega-3 contents of the algae with relatively indigestible cell walls survive passage through the rumen. Algae that may be used in practicing method of the invention include for example; Phaedactylum tricornatum, Spirulina, Chlorella, Nannochloropsis, Monodus subterraneus, Crypthecodinium cohnii, Schizochytrium, Thraustochytrium aggregatum and Ulkenia sp. The algae may be administered as part of the daily feed ration (which can for example be corn, soy alfalfa, straw, wet grain distillers that are by products of the ethanol industry, barley or wheat). In many instances wheat and/or barley are preferred as the regular feedlot ration instead of corn. The algae may be presented in the standard feed ration in dry or wet form or formed and administered as a pellet.

Column 7 of Table 1 presents the additional cost of the amount of algae fed for each protocol per pound of boned beef. In these calculations, for sake of simplification it is assumed that the 1400 pound steer will provide about 400 pounds of boned meat, and ground beef. To maximize the amount of omega 3s DHA plus EPA in the beef at 141 mg costs an additional $0.90 per pound of boned beef. In comparison, the most cost-effective protocol costs only an additional $0.15 per pound of boned beef, but supplies 81 mg of DHA and EPA compared to the 141 mg of the highest algae ration. In effect, for the maximum level of omega 3s, the consumer will be asked to pay 6 times the additional cost to receive 74% more omega 3s.

It is possible that some consumers will prefer to maximize the omega 3s in the beef they buy, and others will look for the most economic offering of a moderate increase in omega 3s.

The specific numbers in the examples provided above may be used generally to describe algae feeding protocols which are smaller and larger than the specific numbers disclosed. There are a near infinite number of algae feeding amounts between 0.05 lb. per day and 0.8 lb. per day that fall within the present invention.

The following working example illustrates that the strong cell walls in schizochytrium and nannochloropsis work to protect the omega-3 fatty acids inside the algae from release in the rumen where it would be hydrogenated and rendered useless in the small intestine.

### Example

Three beef cattle were fed a standard ration of including corn, corn gluten, hay and minerals. A control animal was fed only the standard ration. In addition to the standard ration, a second animal received a slurry of 80% water and 20% nannochloropsis algae (equivalent to 0.2 pounds of algae per day) administered directly into the rumen of the intestine through a cannula. A third animal was fed the standard ration and also received 0.2 pounds of dry schizochytrium algae per day through a cannula directly into the rumen. The animals were fed for 8 days (from December 24 through January 1). December 23 is the day before doing began. On January 1 samples of intestinal solids and fluids were collected using a cannula and then analyzed for combined DHA and EPA content (as a percentage of omega-3 acids present). The omega-3 acids in the rumen fluid are released from broken algal cells. The omega-3 acids in the rumen solids are the acid from intact algal cells that have not been broken. The results of the analysis are shown below in tabular form;

| | 12/23 | 12/29 | 1/1 |
|---|---|---|---|
| % DHA & EPA Rumen Fluid | | | |
| control | 13.2 | 12.5 | 10.2 |
| nanno | 14.4 | 16.5 | 17.3 |
| schizo | 16.4 | 14.2 | 14.8 |
| % DHA & EPA Rumen Solid | | | |
| control | 22.2 | 12.9 | 13.9 |
| nanno | 13.2 | 20.3 | 21.9 |
| schizo | 6.72 | 15.1 | 14.8 |

The results show that schizochytrium and nannochloropsis work in the method of the invention and that schizochytrium had a stronger cell wall than nannochloropsis that worked somewhat better to protect the omega-3 acids in the algae from release in the rumen.

1. The method of increasing the level of omega-3 fatty acids in cattle which comprises feeding the cattle between about 0.05 and 0.8 pounds per day of algae.

2. The method of claim 1 which comprises continuing the feeding for 30 to 90 days.

3. The method of claim 1 which comprises feeding the cattle between about 0.4 and 0.8 pounds per day of algae for 30 to 90 days.

4. The method of claim 2 which comprises feeding the cattle between about 0.2 and 0.4 pounds per day of algae for 30 to 90 days.

5. The method of claim 1 which comprises feeding the cattle algae plus a ration comprising cracked, flaked or steamed corn and other feedlot ingredients.

6. The method of claim 2 which comprises feeding the cattle algae plus a ration comprising cracked, flaked or steamed corn and other feedlot ingredients.

7. The method of claim 3 which comprises feeding the cattle algae plus a ration comprising cracked, flaked or steamed corn and other feedlot ingredients.

8. The method of increasing the level of omega-3 fatty acids in cattle of claim 1 wherein the feeding is conducted for the last 30 to 90 days before the cattle are slaughtered.

9. The method of increasing the level of omega-3 fatty acids in cattle which comprises feeding the cattle between about 0.05 and 3.0 pounds per day of algae and at least one member selected from the group consisting of a low fat grain such as wheat or barley, plus other feedlot ingredients.

10. The method of claim 1 wherein the algae has a call wall capable of protecting the omega-3 fatty acids inside the algae from release in the rumen of the intestine.

11. The method of claim 10 wherein the algae is schizochytrium.

12. The method of claim 1 wherein the algae is nannochloropsis.

13. The method of claim 10 wherein the algae is administered along with at least one of corn, soy alfalfa, straw, wet grain distillers, barley or wheat.

14. The method of claim 13 which comprises feeding the cattle wheat along with the algae.

15. The method of claim 13 which comprises feeding the cattle barley along with the algae.
